(19) United States
(12) Patent Application Publication
(10) Pub. No.: US 2010/0160968 A1
(43) Pub. Date: Jun. 24, 2010

(54) SYSTEMS AND METHODS FOR PEDICLE SCREW-BASED SPINE STABILIZATION USING FLEXIBLE BANDS

(75) Inventors: Abhijeet Joshi, Austin, TX (US); Wayne P. Gray, JR., Pflugerville, TX (US)

Correspondence Address: Sprinkle IP Law Group / Zimmer, 1301 W. 25th Street, Suite 408, Austin, TX, 78705, US

(73) Assignee: Abbott Spine Inc., Austin, TX (US)

(21) Appl. No.: 12/340,366

(22) Filed: Dec. 19, 2008

(51) Int. Cl. A61B 17/70

(52) U.S. Cl. 606/263; 606/246; 606/278; 606/257

(57) ABSTRACT

Embodiments of the disclosure provide a spinal stabilization rod useful for connecting a set of bone fasteners that can anchor a spinal stabilization system onto vertebral bodies. The spinal stabilization rod comprises rigid sections for coupling with bone fastener assemblies engaged in vertebrae and a flexible band for coupling the rigid sections. A bumper may be disposed around the rod to encase the flexible band. The spinal stabilization system may include a dampener.

### TECHNICAL FIELD

This disclosure relates generally to spinal implants, and more particularly to a pedicle screw-based stabilization system which addresses stability and range of motion in the anterior-posterior plane.

### BACKGROUND

The human spine consists of segments known as vertebrae linked by intervertebral disks and held together by ligaments. There are 24 movable vertebrae—7 cervical (neck) vertebrae, 12 thoracic (chest) vertebrae, and 5 lumbar (back) vertebrae. Each vertebra has a somewhat cylindrical bony body (centrum), a number of wing-like projections (processes), and a bony arch. The arches are positioned so that the space they enclose forms the vertebral canal. The vertebral canal houses and protects the spinal cord, and within it the spinal fluid circulates. Ligaments and muscles are attached to various projections of the vertebrae. The bodies of the vertebrae form the supporting column of the skeleton. Fused vertebra make up the sacrum and coccyx, the very bottom of the vertebral column.

The spine is subjected to abnormal curvature, injury, infections, tumor formation, arthritic disorders, and puncture or slippage of the cartilage disks due to variety of reasons. Modern spine surgery often involves the use of spinal stabilization/fixation procedures such as a vertebral body fusion procedure to correct or treat various acute or chronic spine disorders and/or to support the spine. In conjunction with these procedures, some spinal implants may be utilized to help stabilize the spine, correct deformities of the spine such as spondylolisthesis or pseudoarthrosis, facilitate fusion, or treat spinal fractures. Some spinal implants such as a spinal fixation system may provide fused and/or rigid support for the affected regions of the spine. For example, a spinal fixation system may include a corrective spinal implant that is attached to selected vertebrae of the spine by screws, hooks, and clamps. The corrective spinal implant may include spinal rods or plates that are generally parallel to the patient's back. The corrective spinal implant may also include transverse connecting rods that extend between neighboring spinal rods. Spinal fixation systems can be used to correct problems in the cervical, thoracic, and lumbar portions of the spine, and are often installed posterior to the spine on opposite sides of the spinous process and adjacent to the transverse process. Spinal fixation systems when implanted inhibit movement in the affected regions in virtually all directions.

Modern spine surgery often involves spinal fixation through the use of spinal implants or fixation systems to correct or treat various spine disorders or to support the spine. Spinal implants may help, for example, to stabilize the spine, correct deformities of the spine, facilitate fusion, or treat spinal fractures. A spinal fixation system typically includes corrective spinal instrumentation that is attached to selected vertebra of the spine by screws, hooks, and clamps. The corrective spinal instrumentation may include spinal rods or plates that are generally parallel to the patient's back. The corrective spinal instrumentation may also include transverse connecting rods that extend between neighboring spinal rods. Spinal fixation systems are used to correct problems in the cervical, thoracic, and lumbar portions of the spine, and are often installed posterior to the spine on opposite sides of the spinous process and adjacent to the transverse process.

Various types of screws, hooks, and clamps have been used for attaching corrective spinal instrumentation to selected portions of a patient's spine. Examples of pedicle screws and other types of attachments are illustrated in U.S. Pat. Nos. 4,763,644; 4,805,602; 4,887,596; 4,950,269; and 5,129,388. Each of these patents is incorporated by reference as if fully set forth herein.

Often, spinal stabilization systems include rods which can bear a portion of the forces that would otherwise be transmitted along the spine. These rods may be implanted in pairs or in other numbers along portions of the spine of interest. Some spinal stabilization systems may support a portion of the spine including only two vertebrae (and associated anatomical structures) while some spinal stabilization systems support portions of the spine extending beyond two vertebrae. Spinal stabilizations systems can be used to support various portions of the spine, including the lumbar portion of the spine and the thoracic portion of the spine. Regardless of the number of rods implanted, or the portion of the spine in which they may be implanted, the rods can be attached to one or more vertebrae of the spine to provide support and stabilize, align, or otherwise treat the region of the spine of interest. Surgical personnel may use one or more anchor systems to attach the rods to one or more vertebrae. One such anchor system includes pedicle screws constructs which define slots, keyways, grooves, apertures, or other features for accepting and retaining stabilization rods which may be static, dynamic, or a combination of both. In many pedicle screw constructs, pedicle screws are placed in vertebrae selected by surgical personnel.

Often, spinal fixation may include rigid (i.e., in a fusion procedure) support for the affected regions of the spine. Such systems limit movement in the affected regions in virtually all directions (for example, in a fused region). More recently, so called “dynamic” systems have been introduced wherein the implants allow at least some movement of the affected regions in at least some directions, i.e., flexion, extension, lateral, or torsional. Dynamic spinal stabilization systems can better match a patient's anatomy than some spinal stabilization systems used to provide static support. When implanted in a patient, a dynamic spinal stabilization system can allow at least some movement (e.g., flexion, extension, lateral bending, or torsional rotation) of the affected regions of the spine in at least some of the directions, giving the patient a greater range of motion. Dynamic stabilization systems can be used in scenarios in which vertebral body fusion is not desired, in which vertebral body (re)alignment is desired, and in which it is desired to support or strengthen degraded, diseased, damaged, or otherwise weakened portions of the spine.

### SUMMARY

Embodiments disclosed herein can be used as part of a spinal fusion or non-fusion treatment to stabilize the spine and address the omnipresent back pain problem. Specifically, embodiments of a spinal stabilization system may be coupled to and disposed between two pedicle screws to provide flexion and limit extension. Rigid sections may be coupled to pedicle screws, and a flexible band may couple the rigid sections. The flexible band allows the spine to move in flexion, and the rigid sections contact each other to inhibit extension of the spine.

Embodiments disclosed herein may include a rod for stabilizing a portion of a spine. Embodiments of the rod may include a first rigid section having a first selected end, a second rigid section having a second selected end and a flexible band for joining the first rigid section to the second rigid section. In some embodiments, each of the first rigid section and the second rigid section has a cylindrical body for insertion into a bone fastener assembly. In some embodiments, the flexible band limits the separation distance between the first rigid section and the second rigid section when the first rigid section and second rigid section are securely coupled to bone fastener assemblies engaged with vertebrae. In some embodiments, contact between the first rigid section and the second rigid section limits the motion of the first rigid section relative to the second rigid section. In some embodiments, the first rigid section further comprises an aperture for passage of a portion of the flexible band. In some embodiments, the flexible band comprises polyester. In some embodiments, the flexible band comprises Dacron® polyester. In some embodiments, the flexible band is attached to either the first rigid section or the second rigid section before advancement of the flexible band into the patient. In some embodiments, the rod further includes a cap on one or more of the first rigid section and the second rigid section. In some embodiments, one or more of the first rigid section and the second rigid section have an end with a shape formed for selected contact with an end of an adjacent rigid section, wherein the spine has a selected range of motion based on the contact between the end of the first or second rigid section and the end of the adjacent rigid section. In some embodiments, the rod further includes a dampener. In some embodiments, the first rigid section or the second rigid section comprises a cap and one or more spring elements. In some embodiments, the first rigid section or the second rigid section comprises a rigid outer cap bonded to a resilient inner cap, wherein motion of the first rigid section or the second rigid section relative to the bone fastener assembly is controlled by a property of the inner cap. In some embodiments, the first rigid section or the second rigid section comprises an outer polymer sleeve and an inner polymer disc. In some embodiments, the first rigid section or the second rigid section comprises an outer polymer cap defining a space with the first rigid section or the second rigid section and a hydrogel located inside the space.

Embodiments of a spine stabilization system may include a first bone fastener assembly coupled to a first vertebra, a second bone fastener assembly coupled to a second vertebra, and a rod for coupling the first bone fastener assembly to the second bone fastener assembly. The rod may include a first rigid section having a first selected end, a second rigid section having a second selected end and a flexible band for joining the first rigid section to the second rigid section. In some embodiments, each of the first rigid section and the second rigid section has a cylindrical body for insertion into a bone fastener assembly. In some embodiments, the flexible band limits the separation distance between the first rigid section and the second rigid section when the first rigid section and second rigid section are securely coupled to bone fastener assemblies engaged with vertebrae. In some embodiments, contact between the first rigid section and the second rigid section limits the motion of the first rigid section relative to the second rigid section. In some embodiments, the flexible band comprises polyester. In some embodiments, the spine stabilization system includes a dampener coupled to the first rigid section or the second rigid section and coupled to the first bone fastener assembly or the second bone fastener assembly, wherein motion of the first rigid section or the second rigid section relative to the first bone fastener assembly or the second bone fastener assembly is dampened. In some embodiments, motion of the spine is controlled in one or more ranges of motion comprising flexion, extension, lateral bending and torsion.

Embodiments may include a method of stabilizing a portion of the spine. The method may include coupling a first bone fastener assembly to a first vertebra, coupling a second bone fastener assembly to a second vertebra and coupling a rod to the first bone fastener assembly and to the second bone fastener assembly. In some embodiments, the rod has a first rigid section having a first selected end and a second rigid section having a second selected end, and coupling the rod to the first bone fastener assembly or the second bone fastener assembly includes joining the first rigid section to the second rigid section with a flexible band, wherein each of the first rigid section and the second rigid section has a cylindrical body for insertion into a bone fastener assembly. The flexible band limits the separation distance between the first rigid section and the second rigid section when the first rigid section and second rigid section are securely coupled to bone fastener assemblies engaged with vertebrae. Contact between the first rigid section and the second rigid section limits the motion of the first rigid section relative to the second rigid section.

In some embodiments, the method may include comprising mechanically joining the first rigid section to the second rigid section using the flexible band. In some embodiments, the first rigid section or the second rigid section comprises a dampener, wherein coupling the first rigid section to the first bone fastener assembly or coupling the second rigid section to the second bone fastener assembly comprises joining the dampener to the first bone fastener assembly or the second bone fastener assembly. In some embodiments, the method may include coupling one or more cross-link devices to connect two or more rods.

In one embodiment, a first rigid section of the rod may be coupled to a first pedicle screw implanted in a first vertebra and a second rigid section of the rod may be coupled to a second pedicle screw implanted in a second vertebra. The first and second rigid sections may each have a slot, opening or aperture formed therein. Either the first rigid section or the second rigid section may have a flexible band attached thereto, such as by threading the flexible band through an opening and joining two portions of the flexible band to inhibit withdrawing the flexible band from the aperture. Ends of the flexible band may be passed through the aperture in the other rigid section and connected to inhibit withdrawal of the flexible band from the aperture. Once the ends of the flexible band are connected, the spine may have a range of motion that is limited in flexion by the flexible band and that is limited in extension by the rigid sections contacting each other.

Embodiments of a spine stabilization system may be used in a single-level stabilization system, or may be used in a multi-level spine stabilization system, which may provide advantages over multiple levels of the spine.

Other features, advantages, and objects of the disclosure will be better appreciated and understood when considered in conjunction with the following description and the accompanying drawings.

### BRIEF DESCRIPTION OF THE DRAWINGS

A more complete understanding of the present disclosure and the advantages thereof may be acquired by referring to the following description, taken in conjunction with the accompanying drawings in which like reference numbers indicate like features and wherein:

FIG. 1 depicts a simplified graphical representation of a side view showing a portion of a healthy spine and various types of movements of the spine;

FIG. 2A depicts a perspective view of one embodiment of a spinal rod;

FIG. 2B depicts a side view of a spinal stabilization system installed on vertebral bodies according to some embodiments of the disclosure;

FIGS. 3-7 depict views of rigid sections of a spinal rod according to some embodiments of a spine stabilization system;

FIG. 8 depicts a view of one embodiment of a spinal rod;

FIGS. 9A and 9B depict side and cross-section views of one embodiment of a spinal rod according to one embodiment; and

FIGS. 10-13 depict simplified schematic representations of a spinal stabilization rod, according to some embodiments of the disclosure.

### DETAILED DESCRIPTION

The spine stabilization system and the various features and advantageous details thereof are explained more fully with reference to the non-limiting embodiments detailed in the following description. Descriptions of well known starting materials, manufacturing techniques, components and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

A spine is composed of vertebral bones (i.e., vertebrae) stacked up on one another in a smooth alignment. The spinal canal sits within the spinal column and houses the spinal cord and spinal nerves that send signals to and from the brain. The linkages between the vertebrae are soft, with discs in the front and ligaments in the back, allowing displacement and adaptation to stress and load. Due to the unique and complex arrangements and configurations of vertebrae and intervertebral disc components, a healthy spine is strong and flexible and can function through the vigorous demands of daily living, work, and recreational activities. However, the spine is vulnerable to injury and to degeneration, a term which refers to the gradual failure of the spine's biomechanical functions due to aging and wear and tear. The soft tissues (e.g., the intervertebral discs, the ligaments and cartilage of the facet joints) are most vulnerable to degeneration. With normal aging, the discs would gradually collapse and ligaments lose their elasticity and stabilizing ability. Other factors such as those described below may also cause damage to the spine.

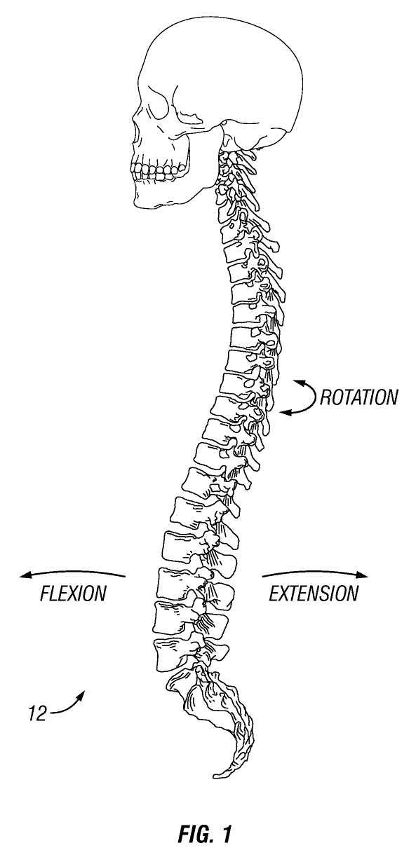

FIG. 1 depicts a simplified graphical representation of a side view of a portion of healthy spine 12 and various types of movements thereof, i.e., flexion, extension and rotation. Various spine deformities, injuries, and the like may cause spine 12 to destabilize or require, resulting in changes to one or more ranges of motion.

Table 1 below lists the ranges of motion for flexion, extension, lateral bending and torsion with normalized values for a normal spine, a destabilized spine, and a spine stabilized with rigid rods.

TABLE 1

| | Healthy | Destabilized | Stabilized with |
|---|---|---|---|
| Range of Motion | Spine | Spine | Rigid Rod(s) |
| Flexion | 1.0 | 1.40-2.80 | 0.2-0.6 |
| Extension | 1.0 | 1.40-2.20 | 0.2-0.6 |
| Lateral Bending | 1.0 | 1.40-2.40 | 0.2-0.7 |
| Torsion | 1.0 | 1.40-2.40 | 0.2-0.8 |

As is known, the biomechanical functions of a healthy spine are very complex and difficult to replicate. There is a continuing need for better spinal implants and implantable devices that can stabilize a weakened/damaged spine and yet simultaneously allow some range of motion so that the patient can enjoy daily life and normal activities without constraints or restrictions.

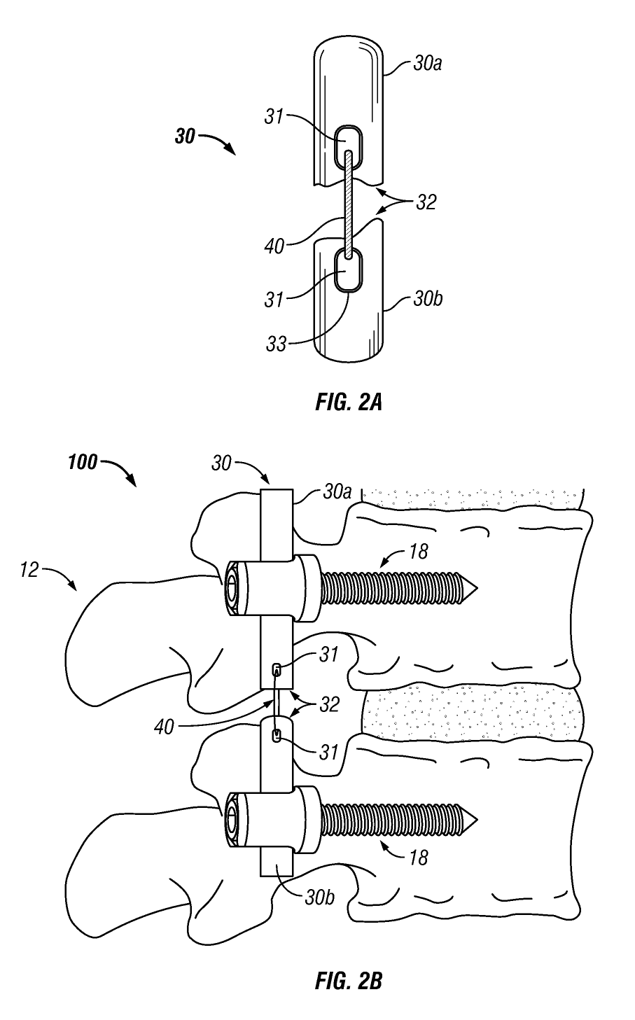

FIG. 2A depicts a perspective view of one embodiment of spinal rod 30 having rigid section 30a and 30b connected by flexible band 40 passing through apertures 31 and further including ends 32 for contact between rigid sections 30a and 30b.

Rigid sections 30a and 30b may be manufactured from a biocompatible material. In some embodiments, rigid sections 30a or 30b are manufactured from PEEK, metal, carbon, composites, ceramics, or the like. Aperture 31 may be formed in rigid section 30a and 30b. In some embodiments, rigid section 30a or 30b may include bushing 33. In some embodiments, bushing 33 may be formed first and placed into an injection molding machine, and material may be injected to form rigid section 30a or 30b around bushing 33. Bushing 33 may be formed from a biocompatible material, steel, titanium, UHMWPE or some other material that can withstand the injection molding process. Bushing 33 may be formed from a material that allows flexible band 40 to move relative to rigid section 30a or 30b.

Rigid sections 30a or 30b may include ends 32 for contact with adjacent rigid sections 30b or 30a. The geometry of ends 32 of rigid sections 30a or 30b may determine the extension range of motion. In some embodiments, the geometry of ends 32 of rigid sections 30a and 30b may act as a mechanical stop or barrier after a desired range of motion is achieved. The geometry of ends 32 of rigid sections 30a and 30b can be designed such that rigid sections 30a and 30b will not affect flexion ROM, but will allow limited extension motion. Ends 32 of rigid sections 30a or 30b may be flat, rounded, or have other shapes. Ends 32 of rigid sections 30a or 30b may be symmetric or asymmetric. In some embodiments, ends 32 of rigid sections 30a or 30b may be designed and matched with one another in various ways to achieve a desired range of motion.

Flexible band 40 may be used to restrict the amount of flexion in spine 12. The ends of flexible band 40 may allow flexible band 40 to pass through apertures 31, bushings 33 or otherwise couple rigid sections 30a and 30b to form rod 30. In some embodiments, flexible band 40 may be manufactured from Dacron® polyester, carbon fiber, Ultra-High Molecular Weight Polyethylene (UHMWPE) fibers, or some other material that can withstand tension exerted on spine 12 by the patient.

FIG. 2B depicts a side view of one portion of spine 12 coupled to spine stabilization system 100. In some embodiments, spine stabilization system 100 may include bone fastener assemblies 18 and rods 30.

Each bone fastener assembly lo may include a bone screw or other bone fastener, a collar, a ring, and other components for coupling rod 30 to a vertebra. Bone screws may have a head for engagement by a driver or other tool and a shank for advancement into a vertebra. A bone screw may be advanced into the patient via an extender sleeve. The bone screw may be positioned relative to the pedicle of a vertebra. A tool may be advanced into the patient and engage the head of the bone screw. For example, a driver may be advanced into the patient via the extender sleeve and engage the head of a bone screw. The driver may rotate the head of the bone screw to advance the bone screw into the vertebra.

A collar, ring, or other component may be advanced into the patient and connected with the bone screw to form bone fastener assembly 18. A collar may be used to couple rod 30 to a bone screw. Rod 30 may be positioned in the collar to couple rod 30 to bone fastener assembly 18. Each rod 30 may include rigid sections 30a and 30b, flexible band 40, apertures 31 and ends 32 having selected surfaces or geometries for contact with other ends 32. In some embodiments, rigid sections 30a and 30b of rods 30 may be positioned in collars of bone fastener assemblies 18 prior to assembly of bone fastener assembly 18. A ring may be used to provide a range of motion of a collar relative to the head of the bone screw. Closure members may be used to secure rod 30 in collars of bone fastener assemblies 18 coupled to spine 12.

Rigid sections 30a or 30b may be advanced into the patient and positioned in bone fastener assemblies 18. Advancement of rigid sections 30a and 30b into a patient may be accomplished by attaching rigid sections 30a or 30b to a tool and advancing the end of the tool into the patient, by advancing a tool into the patient and then using the tool to guide the advancement of rigid sections 30a or 30b, or by engaging rigid section 30a or 30b with another component (e.g. bone fastener assembly 18, a cross-link device, etc.) and then advancing the component and rigid section 30a or 30b into the patient.

Rigid sections 30a and 30b may be connected using flexible band 40 to form part of spine stabilization system 100. In some embodiments, rigid sections 30a and 30b may be coupled to bone fastener assemblies 18 and flexible band may be passed through one or more apertures 31 in rigid sections 30a or 30b and joined to inhibit withdrawal of flexible band from either rigid section 30a or 30b.

Surgical techniques to stabilizing the spine may include implanting bone screws in pedicles or otherwise coupling bone fastener assemblies 18 to vertebrae and coupling rods 30 to bone fastener assemblies 18 to provide a desired level of movement of spine 12.

The use of pedicle screws in spine stabilization is generally known in the art. Techniques of implanting the bone screws, both invasive and minimally-invasive (MIS) are familiar to most spine surgeons. Once bone screws are engaged with selected vertebrae, a rod may be coupled to the bone screws. One technique of coupling a rod to a spine involves implanting pedicle screws in pedicles and coupling a rod between the implanted pedicle screws. The patient may be positioned on a surgical table, implantation sites may be identified, bone screws may be advanced into the patient and implanted in the vertebrae, and a rod may be coupled to the bone screws. Tools, such as an awl, tap, sleeve, driver, and the like may be used to prepare a vertebra for implantation, to guide or position the pedicle screw at the implantation site, to implant the pedicle screw into the bone, and the like. U.S. Pat. No. 7,250,052 describes at least one method for implanting bone screws in vertebrae and is incorporated herein by reference.

Once the bone screws are implanted in the vertebrae, other portions of spine stabilization system 100 may be advanced into the patient. One embodiment of a method for stabilizing the spine may include advancing first rigid section 30a of rod 30 into the patient and coupling first rigid section 30a to first bone fastener assembly 18. In some embodiments, first rigid section 30a may be coupled to flexible band 40 prior to advancement into the patient. In some embodiments, first rigid section 30a may be positioned in a collar of bone fastener assembly 18 and secured using a closure member. In some embodiments, first rigid section 30a may be positioned in a collar and held in place by a tool coupled to the bone screw, the collar, both, or some other portion or component of bone fastener assembly 18. Once first rigid section 30a is positioned in first bone fastener assembly 18, second rigid section 30b may be advanced into the patient. In some embodiments, second rigid section 30b may be coupled to flexible band 40 prior to advancement into the patient. In some embodiments, second rigid section 30b may be positioned in bone fastener assembly 18 and secured using a closure member. In some embodiments, second rigid section 30b may be positioned in a collar in bone fastener assembly 18 and held in place by a tool coupled to the bone screw, the collar, both, or some other portion or component of bone fastener assembly 18. Once first rigid section 30a and second rigid section 30b are positioned in bone fastener assemblies 18, the ends of flexible band 40 may be passed through apertures 31 in one or both rigid sections 30a and 30b. After band 40 is passed through apertures 31 in rigid sections 30a and 30b, band 40 may be tightened or otherwise configured to have a desired length or tension. In some embodiments, after band 40 has been passed through apertures 31 in rigid sections 30a and 30b, the ends of band 40 may be joined to each other or rigid sections 30a or 30b to inhibit withdrawal of either end from first rigid section 30a or second rigid section 30b. In some embodiments, joining the ends of flexible band 40 may include mechanically, chemically, or thermally joining the ends. For example, mechanically joining the ends may include tying the ends together or to rigid sections 30a or 30b, crimping the ends together or to a feature of rigid sections 30a or 30b, or the like. Chemically configuring the ends may include using an adhesive to bond the ends together or to rigid sections 30a or 30b. In some embodiments, rigid section 30a or 30b may have a feature for connection to the ends of flexible band 40.

Once rod 30 has been assembled and rigid sections 30a and 30b have been secured to bone fastener assembly 18, extender sleeves and other tools may be withdrawn from the patient. After implantation of spine stabilization system 100, contact between ends 32 of rigid sections 30a and 30b may control or limit extension in the spine and flexible band 40 may control or limit flexion in the spine. The surgeon may decide the gap between rigid sections of rod 30, depending upon the range of motion and patient's spine stability, by adjusting the length of flexible band 40 between rigid sections 30a and 30b.

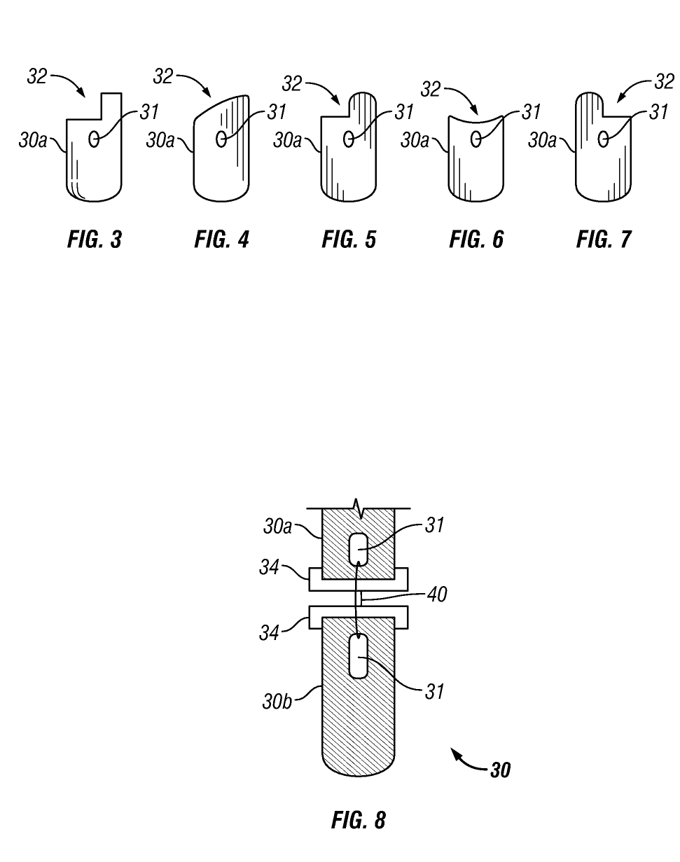

FIGS. 3-7 depict views of embodiments of rigid sections 30a or 30b. In some embodiments, rigid section 30a or 30b may be symmetric to allow unbiased movement of the spine, may be asymmetric to bias the spine in a preferred orientation, may include features to restrict movement in a direction or about an axis, or may have some other feature or shape to promote a healthy spine.

FIG. 8 depicts a side cross-section view of one embodiment of rod 30 having caps 34 on ends 32 of rigid sections 30a and 30b. Caps 34 may be useful to avoid hard contact between rigid sections 30a and 30b. In some embodiments, caps 34 may provide a desired cushion effect between adjacent rigid sections 30a and 30b.

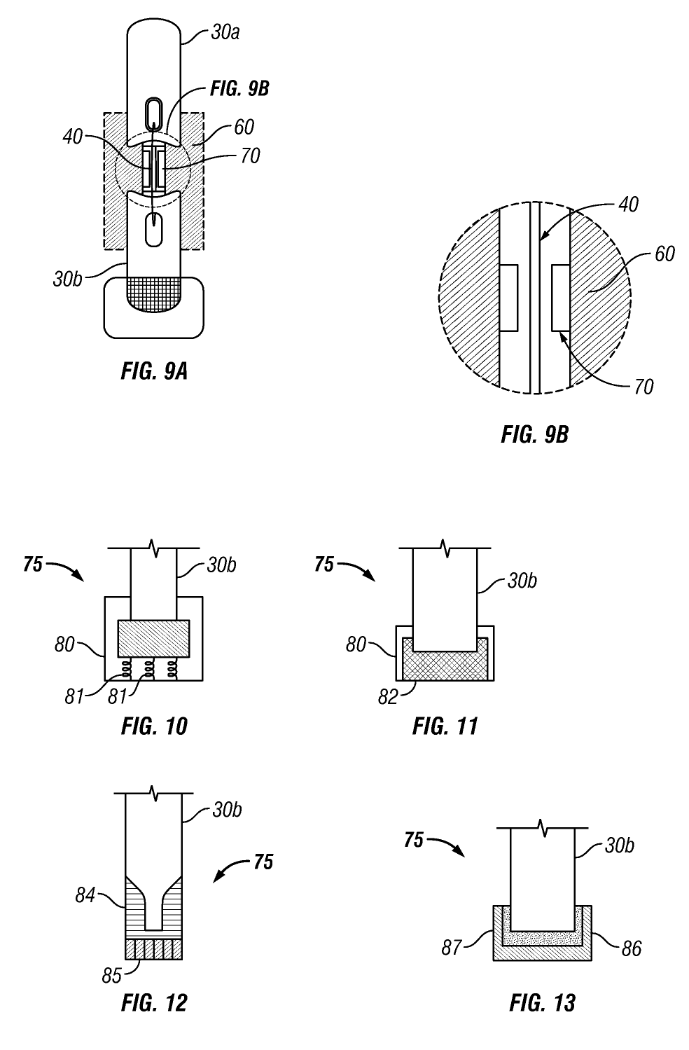

In some embodiments, spinal stabilization system 100 may include other components for controlling motion of a spine. FIGS. 9A and 9B depict views of a portion of one embodiment of spine stabilization system 100 including rigid sections 30a and 30b, flexible band 40 and casing 60.

Casing 60 may surround flexible band 40 and isolate flexible band 40 from contact with other components of spine stabilization system 100, anatomic structures, debris, or to contain wear particles of spine stabilization system 100, or the like. In some embodiments, casing 60 may surround flexible band 40 and extend a desired distance along rigid sections 30a and 30b to isolate flexible band 40. Casing 60 may allow stretching of band 40. This casing may be manufactured from biocompatible materials such as polyurethane (PU) and its co-polymeric configurations.

In some embodiments, spine stabilization system 100 may include bushing 70. Bushing 70 may be manufactured from metals, metal alloys, ceramics, composites, polymers, or some other material. The presence of bushing 70 may reduce or minimize the friction between the rigid rod and flexible band. In some embodiments, bushing 70 may be manufactured from a material that can withstand an injection molding process. Bushing 70 may be inserted into an injection molding device and material may be injected into the device to surround bushing 70 and form casing 60. Bushing 70 may reduce friction between flexible band 40 and aperture 31 in rigid section 30a or 30b.

Spine 12 may benefit from dampened motion. In some embodiments, rod 30 may include dampeners 75 to provide dampened motion to spine 12. FIGS. 10-13 depict cross-sectional views of embodiments of portions of rigid sections 30a or 30b including dampener 75. As depicted in FIG. 10, in some embodiments, dampener 75 may include cap 80 and one or more spring elements 81. Cap 80 may be able to move relative to rigid section 30a or 30b. Spring elements 81 may have a first end attached to cap 80 and a second end attached to a second end of rigid section 30a or 30b.

In some embodiments, rigid section 30a or 30b having dampener 75 may be coupled to bone fastener assembly 18. In one embodiment, cap 80 may be coupled to bone fastener assembly 18 and rigid section 30a or 30b may be able to move with dampened motion relative to bone fastener assembly 18 such that motion of rigid section 30a or 30b. The range of motion or degree of dampening may be controlled by one or more of the spring stiffness properties, length of spring elements 81, the number of spring elements 81, the angle of attachment of spring elements 81 to cap 80 or rigid section 30a or 30b, and the like.

As depicted in FIG. 11, in some embodiments, dampener 75 may include outer cap 80 and inner cap 82. Outer cap 80 may be manufactured from a rigid material such as titanium, titanium alloys, stainless steel, or some other metal. Inner cap 82 may be manufactured from a resilient material such as a polymer. Inner cap 82 may be bonded to outer cap 80.

In some embodiments, rigid section 30a or 30b may be coupled to bone fastener assembly 18. Cap 80 may be coupled to bone fastener assembly 18 such that rigid section 30b is able to move with dampened motion relative to bone fastener assembly 18. Motion of rigid section 30a or 30b may be possible due to compression and strain on resilient material used to manufacture inner cap 82. The range of motion or degree of dampening may be controlled by one or more of the viscosity of the polymer, elastic yield strength, and other properties or characteristics of polymer cap 82.

As depicted in FIG. 12, in some embodiments, dampener 75 may include polymer sleeve 84 and polymer disc 85. Rigid section 30a or 30b may have an end formed with a tapered profile, stepped profile, or other narrowing profile such that rigid section 30a or 30b and polymer sleeve 84 may have the same outer profile as other portions of rod 30. Polymer sleeve 84 and rigid section 30a or 30b may be coupled to bone fastener assembly 18 such that motion of rigid section 30a or 30b relative to bone fastener assembly 18 is dampened by polymer sleeve 84 or polymer disc 85 or both.

In some embodiments, rigid section 30a formed with polymer sleeve 84 and polymer disc 85 may be inserted into bone fastener assembly 18. The interaction between polymer sleeve 84 and polymer disc 85, the material composition of polymer sleeve 84 or polymer disc 85, or some combination may determine the dampening characteristics of rigid section 30a or 30b.

As depicted in FIG. 13, in some embodiments, rod 30 may include dampener 75 in which polymer cap 86 is coupled to rigid section 30a or 30b and forming a space therein and a polymer injected therein. Polymer cap 86 may be formed from polyurethane or some other soft polymer. In some embodiments, hydrogel 87 may be injected in liquid form at room temperature into the space formed by polymer cap 86 and rigid section 30a or 30b, and the hydrogel will solidify once inside the human body at elevated temperature of 37° C. Polymer cap 86 may be coupled to bone fastener assembly 18 such that motion of rigid section 30a or 30b relative to bone fastener assembly 18 is dampened by polymer cap 86 or hydrogel 87 or both. The range of motion or degree of dampening may be controlled by the properties of polymer cap 86 or hydrogel 87, the interaction between polymer cap 86 and hydrogel 87, or some combination.

In some embodiments, polymer casing 60 and dampening element 75 may provide a vehicle for the delivery of drugs. A drug dosage may be disposed in spine stabilization system and be released after specific time intervals in a controlled manner.

In some embodiments, an advantage to having rigid sections 30a or 30b with dampener 75 may be the extra cushioning effect, which may be particularly noticeable in discectomy patients.

Another advantage to embodiments of spine stabilization system 100 is that a reasonable range of motion in either lateral or torsional motion may be possible based on one or more components of spine stabilization system 100. In some embodiments, the anterior-posterior range of motion are a function of flexible band properties, properties of external polymer casing 60, and stiffness of dampener element 75. Lateral and torsional range of motion may primarily be a function of properties of lower rigid section 30a or 30b.

Another advantage to embodiments of spine stabilization system 100 may be the capability to be used in conjunction with other non-fusion devices, such as nucleus replacements and facet joint replacements.

Another advantage may be the capability for embodiments of spine stabilization system 100 to be implanted using minimally invasive surgery (MIS) techniques and used with existing pedicle screw platforms. In the event that fusion between vertebrae is required, embodiments may be easily removed and replaced with other rods.

In some embodiments, spinal stabilization system 100 may include additional rods positioned further superior or inferior along spine 12, with the additional rods being anisotropic spinal stabilization rods, dynamic stabilization rods, non-dynamic rods, and/or rigid rods. Within this disclosure, the term “dynamic” refers to the flexing capability of a spinal rod. It should be understood that spinal stabilization system 100 may also include suitable transverse rods or cross-link devices that help protect the supported portion of spine 12 against torsional forces or movement. Some possible examples of suitable cross-link devices are shown in co-pending U.S. patent application Ser. No. 11/234,706, filed on Nov. 23, 2005 and naming Robert J. Jones and Charles R. Forton as inventors (the contents of this application are incorporated fully herein by reference). Other known cross-link devices or transverse rods may also be employed.

In the foregoing specification, specific embodiments have been described with reference to the accompanying drawings. However, as one skilled in the art can appreciate, embodiments of the anisotropic spinal stabilization rod disclosed herein can be modified or otherwise implemented in many ways without departing from the spirit and scope of the disclosure. Accordingly, this description is to be construed as illustrative only and is for the purpose of teaching those skilled in the art the manner of making and using embodiments of an anisotropic spinal stabilization rod. It is to be understood that the embodiments shown and described herein are to be taken as exemplary. Equivalent elements or materials may be substituted for those illustrated and described herein. Moreover, certain features of the disclosure may be utilized independently of the use of other features, all as would be apparent to one skilled in the art after having the benefit of this description of the disclosure.

What is claimed is:

1. A rod for stabilizing a portion of a spine, comprising:
a first rigid section having a first selected end;
a second rigid section having a second selected end; and
a flexible band for joining the first rigid section to the second rigid section,
wherein each of the first rigid section and the second rigid section has a cylindrical body for insertion into a bone fastener assembly;
wherein the flexible band limits the separation distance between the first rigid section and the second rigid section when the first rigid section and second rigid section are securely coupled to bone fastener assemblies engaged with vertebrae, and
wherein contact between the first rigid section and the second rigid section limits the motion of the first rigid section relative to the second rigid section.

2. The rod of claim 1, wherein the first rigid section further comprises an aperture for passage of a portion of the flexible band.

3. The rod of claim 1, wherein the flexible band comprises polyester.

4. The rod of claim 3, wherein the flexible band comprises Dacron® polyester.

5. The rod of claim 1, wherein the flexible band is attached to either the first rigid section or the second rigid section before advancement of the flexible band into the patient.

6. The rod of claim 5, further comprising a cap on one or more of the first rigid section and the second rigid section.

7. The rod of claim 1, wherein one or more of the first rigid section and the second rigid section have an end with a shape formed for selected contact with an end of an adjacent rigid section, wherein the spine has a selected range of motion based on the contact between the end of the first or second rigid section and the end of the adjacent rigid section.

8. The rod of claim 1, further comprising a dampener.

9. The rod of claim 8, wherein the first rigid section or the second rigid section comprises a cap and one or more spring elements.

10. The rod of claim 8, wherein the first rigid section or the second rigid section comprises a rigid outer cap bonded to a resilient inner cap, wherein motion of the first rigid section or the second rigid section relative to the bone fastener assembly is controlled by a property of the inner cap.

11. The rod of claim 8, wherein the first rigid section or the second rigid section comprises an outer polymer sleeve and an inner polymer disc.

12. The rod of claim 8, wherein the first rigid section or the second rigid section comprises an outer polymer cap defining a space with the first rigid section or the second rigid section and a hydrogel located inside the space.

13. A system for stabilizing a portion of the spine, comprising:
a first bone fastener assembly coupled to a first vertebra;
a second bone fastener assembly coupled to a second vertebra; and
a rod for coupling the first bone fastener assembly to the second bone fastener assembly, the rod comprising:
a first rigid section having a first selected end;
a second rigid section having a second selected end; and
a flexible band for joining the first rigid section to the second rigid section,
wherein each of the first rigid section and the second rigid section has a cylindrical body for insertion into a bone fastener assembly;
wherein the flexible band limits the separation distance between the first rigid section and the second rigid section when the first rigid section and second rigid section are securely coupled to bone fastener assemblies engaged with vertebrae, and
wherein contact between the first rigid section and the second rigid section limits the motion of the first rigid section relative to the second rigid section.

14. The spine stabilization system of claim 13, wherein the flexible band comprises polyester.

15. The spine stabilization system of claim 13, farther comprising a dampener coupled to the first rigid section or the second rigid section and coupled to the first bone fastener assembly or the second bone fastener assembly, wherein motion of the first rigid section or the second rigid section relative to the first bone fastener assembly or the second bone fastener assembly is dampened.

16. The spine stabilization system of claim 15, wherein motion of the spine is controlled in one or more ranges of motion comprising flexion, extension, lateral bending and torsion.

17. A method of stabilizing a portion of the spine, comprising:
coupling a first bone fastener assembly to a first vertebra;
coupling a second bone fastener assembly to a second vertebra;
coupling a rod to the first bone fastener assembly and to the second bone fastener assembly, wherein the rod comprises:
a first rigid section having a first selected end; and
a second rigid section having a second selected end;
joining the first rigid section to the second rigid section with a flexible band,
wherein each of the first rigid section and the second rigid section has a cylindrical body for insertion into a bone fastener assembly;
wherein the flexible band limits the separation distance between the first rigid section and the second rigid section when the first rigid section and second rigid section are securely coupled to bone fastener assemblies engaged with vertebrae, and
wherein contact between the first rigid section and the second rigid section limits the motion of the first rigid section relative to the second rigid section.

18. The method of claim 17, comprising mechanically joining the first rigid section to the second rigid section using the flexible band.

19. The method of claim 17, wherein the first rigid section or the second rigid section comprises a dampener, wherein coupling the first rigid section to the first bone fastener assembly or coupling the second rigid section to the second bone fastener assembly comprises joining the dampener to the first bone fastener assembly or the second bone fastener assembly.

20. The method of claim 17, further comprising coupling one or more cross-link devices to connect two or more rods.
